(19) United States
(12) Patent Application Publication
(10) Pub. No.: US 2012/0207792 A1
(43) Pub. Date: Aug. 16, 2012

(54) INJECTABLE FILLER FOR PODIATRIC AND ORTHOPEDIC USES

(76) Inventor: Ayman Boutros, Great Falls, VA (US)

(21) Appl. No.: 13/349,873

(22) Filed: Jan. 13, 2012

Related U.S. Application Data
(60) Provisional application No. 61/432,930, filed on Jan. 14, 2011.

(51) Int. Cl. A61K 31/78; A61K 31/785; A61K 9/14; A61P 43/00; A61P 25/00

(52) U.S. Cl. 424/400; 424/78.31

(57) ABSTRACT

A podiatric filler composition comprising a pliable biocompatible material and a physiologically acceptable suspending agent. The podiatric filler compositions may be used in methods for treating fat pad atrophy, foot pain, plantar faciitis, metatarsalgia, injury, and rheumatoid arthritis.

This application is a U.S. nonprovisional application based on U.S. Provisional Application No. 61/432,930 filed on Jan. 14, 2011.

### FIELD OF THE INVENTION

The present invention relates to compositions comprising a pliable biocompatible material and a physiological acceptable suspending agent for use for in the treatment of podiatric conditions, methods of manufacture and uses thereof.

### BACKGROUND OF THE INVENTION

The human body contains cushioning pads lying beneath the dermal layer that serve as shock absorbers to protect bones, tendons, and other anatomical structures. These cushioning pads are commonly referred to as fat pads (or fatty pads) because they primarily contain fatty tissue comprising a mixture of fatty acids (e.g., 1.6% myristate, 13.6% palmitate, 1.5% stearate, 10.6% palmitoleate, 4.1% vaccenate, 40.6% oleate, and 14.6% linoleate.) See Buschmann, et al. Foot & Ankle, 14: 389-394, 1993; See also U.S. Pat. No. 6,989,373. Three distinct fat pads (or corpus adiposum) are located below the plantar surface of the foot, at the three contact points with the ground, under the heel, on the metatarsal head, and on the outer arch contact area. See FIG. 1. Septa in the foot fat pads form a trabecular network of intercommunicating chambers. The intercommunicating chambers are disposed in whorls that follow the curvature and torsion of the calcaneus (heel bone). See Blechschmidt, E., Die Architektur des Fersenpolsters. Gegenbaurs Morphologisches Jarhbuch, 73: 20-68, 1934 (translated and re-edited as: Blechschmidt, E., The Structure of the Calcaneal Padding. Foot & Ankle, 2: 260-283, 1982). The fat pads can be damaged, displaced, stretched, or thinned out by injury, disease and/or the natural aging process so that they no longer provide adequate shock absorbency (“fat pad atrophy”). The risk of fat pad atrophy increases if the individual is overweight, has diabetes, engages in athletics, or has often worn thin-sole or high-heel shoes. See D'Ambrosia, Orthopedics, 10:137-142, 1987; and Jahss, et al., Foot & Ankle, 13: 227-232, 1992 and Alexander, et al., Journal of Zoology—London, A209: 405-419, 1986. Also, cortisone injections (e.g., for the treatment of plantar fasciitis) may cause fat pad atrophy. See D'Ambrosia, Orthopedics, 10:137-142, 1987; Buschmann, et al., Foot & Ankle, 16: 254-258, 1995.

Fat pad atrophy, either directly or by aggravated by another condition leads to painful podiatric pathologies that can make it difficult or impossible to walk, exercise, or wear certain footwear (e.g., high heeled shoes.) See Narvaez, et al., Radiographics, 20: 333-352, 2000.

Progressive orthopedic foot deformities, such as pes cavus, digitii flexus, and equinous deformities, may result in fat pad atrophy. Systemic diseases such as rheumatoid arthritis also cause deformities that shift the fat pad under the metatarsal heads or change the composition to exhibit an increased fat viscosity that decreases the ability of the heel to absorb and dissipate the energy generated during ambulation. See Resnick, et al. (August 1999) Foot Ankle Int. 29(8): 481-4. Further, a persons natural foot structure may lead to podiatric problems. For example, if the arch of the foot is weak or the balls of the feet are thin, a person typically feels extra pressure around the base of their foot and is often restricted to flat shoes for comfort.

Certain footwear can create or aggravate painful podiatric pathologies. For instance, wearing a high heeled shoe changes the foot anatomy and places pressure on the heads of the metatarsals rather than the base. The metatarsal bones rest on this fat pad in the normal standing position, but when the foot is put into a high heeled shoe the position of the metatarsal heads and the fat pad changes. The fat pad is shifted backwards and metatarsal heads are then placed in a downward slope with the heads taking more weight-bearing than normal. This creates discomfort after a short time since there is less effective padding in this region because the fat pad is shifted backward. Morton's Neuroma is the most typical form of foot neuralgia caused by wearing high heeled shoes.

The ageing and active segments of the population are especially affected by fat pad atrophy. With the normal loss of fat pads, pressure area starts developing over the metatarsal and the heel area as early as age 30 leading to foot pain (e.g., algesia and hyperalgesia). D'Ambrosia, Orthopedics, 10:137-142, 1987.

Individuals suffering from fat pad atrophy currently rely on orthoses and in-sole cushioning. However, these therapies fail to address the fundamental problem of the lack of fad pad cushioning. Thus be highly desirable to develop a method for restoring the thickness of the fat pads, and consequently their cushioning function.

For instance, collagen injections into the ball of the foot can augment the fat pad, but collagen injections provide only transient relief (e.g., 6 months). The Foot-Tuck Fat Pad Augmentation (Beverly Hills Aesthetic Foot Surgery) [2010]. See also U.S. Pat. No. 6,989,373. Additionally, its effectiveness has been questioned by medical professionals. Moreover, this procedure may produce an immune response (immediate or delayed), may cause allergic reactions, or have side-effects (e.g., clumping, lumping, nodule formation, and granuloma formation.)

Another approach suggests the transplantation of autologous fat into the patient's foot fat pad, but this system suffers from an inherently limited source of material. The Foot-Tuck Fat Pad Augmentation (Beverly Hills Aesthetic Foot Surgery) [2010].

Other approaches to treat fat pad atrophy involve the use of a regenerative tissue matrix, GRAFTJACKET® (biological tissue for human implantation) that contains a matrix of intact connective tissue (e.g., collagen, laminen, and elastin fibers) and preserved vascular channels that act as a scaffold to allow the cells to migrate and grow in the matrix. See U.S. Pat. Nos. 5,336,616; 5,024,830; and 4,865,87; and Brigido, et al. (Jan. 1, 2004) OrthoSuperSite®. However, the long-term efficacy of this product is still unclear, as is whether it restores the normal physiological function of the fat pad.

Plantar injections of silicone fluid have been used to relieve localized pressure-related foot disorders, such as corns and calluses and to reduce risk factors for ulceration in diabetic's foot. Balkin, Fluid silicone implantation of the foot. In Neale's common foot disorders: diagnosis and management. 5th ed. Lorimier, Churchill Livingstone, U.K., 387-400, 1997; Van Schie, et al., Diabetes Care, 23: 634-638, 2000; and Crews, et al., Journal of Biomedical Materials Research, 93B(1): 227-235, Jan. 20, 2010. The injected silicone is engulfed and retained within histocyte cell body as microscopic droplets where it stimulates the local deposition of collagen fibers and thickens the skin at the site of injection by inducing the local formation of scar-like fibrous tissue. However, silicone is not compatible with the normal fatty acid composition, and does not participate in restoring the normal physiological function of the fat pad.

The foregoing approaches generally provide only short term relief and thus require frequent repeat injections which is both expensive and inconvenient for patients. Accordingly, there is a demand for a durable, long-lasting, biocompatible approach that restores the normal physiological function of the fat pad.

### SUMMARY OF THE INVENTION

The present invention provides a podiatric filler composition comprising a pliable biocompatible material and a physiologically acceptable suspending agent.

One embodiment of the invention provides a method for treating or preventing a condition in a subject's foot comprising injecting into a fat pad of the subject's foot an effective amount of a composition comprising an unsubstituted acrylate/substituted acrylate copolymer.

In another embodiment, the invention provides for a method for treating or preventing a condition in a subject's foot comprising injecting into a fat pad of the subject's foot an effective amount of a composition comprising a copolymer of phenylethyl acrylate and phenylethyl methacrylate.

In another embodiment, the invention provides for a method for supplementing a fat pad of a subject comprising injecting into a fat pad of the subject an effective amount of a composition comprising an unsubstituted acrylate/substituted acrylate copolymer.

In a further embodiment, the invention provides for a method of preparing a composition for treating or preventing a condition in a subject's foot comprising admixing a finely ground copolymer of phenylethyl acrylate and phenylethyl methacrylate with a physiologically acceptable suspending agent to form an injectable, biocompatible material.

In another embodiment, the condition is foot pain, fat pad atrophy, progressive orthopedic foot deformity. In a further embodiment, the progressive orthopedic foot deformity is pes cavus, digitii flexus, equinous deformity, or any combination thereof. In another embodiment, the subject has a systemic disease including but not limited to rheumatoid arthritis and diabetes.

In a still further embodiment, the invention provides for the use of a composition comprising an effective amount of an unsubstituted acrylate/substituted acrylate copolymer and a physiologically acceptable suspending agent for treating fat pad atrophy. Another embodiment of the invention is a composition for treating fat pad atrophy comprising an effective amount of an unsubstituted acrylate/substituted acrylate copolymer and a physiologically acceptable suspending agent.

In a still further embodiment, the invention provides for the use of a composition comprising an effective amount of an proteoglycan and a physiologically acceptable suspending agent for treating fat pad atrophy. Another embodiment of the invention is a composition for treating fat pad atrophy comprising an effective amount of a proteoglycan and a physiologically acceptable suspending agent. In another embodiment, the proteoglycan is aggrecan, decorin, biglycan, fibromodulin, lumican, keratocan, epiphycan, or osteoglycin.

One embodiment of the invention provides for a method for treating or preventing a condition in a subject's foot comprising injecting into a fat pad of the subject's foot an effective amount of a composition comprising an unsubstituted acrylate/substituted acrylate copolymer. In one embodiment, the condition is fat pad atrophy or a progressive orthopedic foot deformity. In another embodiment, the progressive orthopedic foot deformity is pes cavus, digitii flexus, equinous deformity or any combination thereof. In one embodiment, the condition is a shift of the fat pad under the metatarsal heads. In a further embodiment, the shift of the fat pad under the metatarsal heads is caused by the subject wearing high heeled shoes. In a further embodiment, the subject has a systemic disease. In another embodiment, the systemic disease is rheumatoid arthritis or diabetes. The invention also provides for use of a composition comprising an effective amount of an unsubstituted acrylate/substituted acrylate copolymer and a physiologically acceptable suspending agent for treating fat pad atrophy.

In one embodiment, the invention provides for a method for supplementing a fat pad of a subject comprising injecting into a fat pad of the subject an effective amount of a composition comprising an unsubstituted acrylate/substituted acrylate copolymer. In another embodiment, the substituted acrylate monomer of the unsubstituted acrylate/substituted acrylate copolymer is substituted with a methyl group. In another embodiment, the substituted acrylate monomer is substituted with a hydrocarbon chain of two, three, four or five carbons. In another embodiment, the substituted acrylate monomer is substituted with a halogen group. In further embodiment, wherein the substituted acrylate monomer is substituted with a nitrile group. In further embodiment, the unsubstituted acrylate/substituted acrylate copolymer is a copolymer of phenylethyl acrylate and phenylethyl methacrylate. In still further embodiment, the copolymer is cross-linked with 1.4 butanediol diacrylate.

In one embodiment, the invention provides for a method of preparing a composition for treating or preventing a condition in a subject's foot comprising admixing a finely ground copolymer of phenylethyl acrylate and phenylethyl methacrylate with a physiologically acceptable suspending agent to form an injectable, biocompatible material.

In one embodiment, the invention provides for a method of preparing a soft tissue filler for a fat pad of a subject comprising admixing an acrylate/methacrylate copolymer with a physiologically acceptable suspending agent to form an injectable, biocompatible material.

In one embodiment, the unsubstituted acrylate/substituted acrylate copolymer comprises particles in a suspension. In another embodiment, the particles have a diameter of less than about 0.01, 10, or 100 μm. In a further embodiment, the particles have a diameter of about 0.01 μm to about 10 μm or about 0.1 μm to about 5 μm.

In one embodiment, the substituted acrylate monomer of the unsubstituted acrylate/substituted acrylate copolymer is substituted with a methyl group. In another embodiment, the substituted acrylate monomer is substituted with a hydrocarbon chain of two, three, four or five carbons. In a further embodiment, the substituted acrylate monomer is substituted with a halogen group, a nitrile group, or a combination thereof.

In one embodiment, the acrylate/methacrylate copolymer is a finely ground solid. In another embodiment, the finely ground solid is a powder, a non-porous microbead, or a microsphere. In a further embodiment, the finely ground solid has a diameter of from about less than 0.1 μm to about 10 μm. In a further embodiment, the solid comprises particles each having a diameter of: about 10 μm to about 100 μm; about 0.01 μm to about 10 μm, about 0.01 μm to about 5 μm, or any combination thereof.

In another embodiment, the suspension has a viscosity of about 100 to about 1,000 mPa·S.

In one embodiment, the method further comprising administering an anti-inflammatory agent. In another embodiment, the anti-inflammatory agent is a non-steroidal anti-inflammatory drug (NSAID). In a further embodiment, the anti-inflammatory agent is acetaminophen, azapropazone, diclofenac, diflunisal, etodolac, fenbufen, fenoprofen, flurbiprofen, ibuprofen, indomethacin, ketoprofen, ketorolac, mefenamic, meloxicam, nabumetone, naproxen, phenylbutazone, piroxicam, a salicylate, sulindac, tenoxicam, tiaprofenic acid, or tolfenamic acid. In still further embodiment, the salicylate is acetylsalicylic acid, amoxiprin, benorylate, choline magnesium salicylate, ethenzamide, faislamine, methyl salicylate, magnesium salicylate, salicyl salicylate, or salicylamide.

In one embodiment, the method further comprising administering an analgesic. In another embodiment, the analgesic is acetaminophen, amitriptyline, carbamazepine, codeine, dihydromorphine, fentanyl patch, Flupirtine, fluriprofen, gabapentin, hydrocodone APAP, hydromorphone, ibuprofen, ketoprofen, morphine, oxycodone, pentazocine, pethidine, phenacetin, pregabalin, propoeylphene, propoyl APA, salicylamide, tramadol, tramadol APAP, or voltaren.

In one embodiment, the method further comprises administering a local anesthetic. In another embodiment, the local anesthetic is amethocaine, articaine, benzocaine, bupivacaine, mepivacaine, cocaine, cinchocaine, chloroprocaine, cyclomethycaine, dibucaine, dimethocaine, EMLA® (eutectic mixture of lidocaine and prilocalne), etidocaine, larocaine, levobupivacaine, lidocaine, lignocaine, procaine, piperocaine, prilocalne, proparacaine, propoxycaine, ropivacaine, saxitoxin, tetracaine, tetrodotoxin, or trimecaine.

In another embodiment, the molar ratio between the phenylethyl acrylate and the phenylethyl methacrylate is selected from the group consisting of: between about 1:1000 and about 1:1; between about 1:10 and about 1:1; between about 1:1000 and about 1:10; about 1:1000; about 1:100; about 1:10; about 1:9; about 1:8; about 1:7; about 1:6; about 1:5; about 1:4; about 1:3; about 1:2; about 1:1; between about 1000:1 and about 1; between about 10:1 and about 1:1; between about 1000:1 and about 10:1; about 1000; about 100:1; about 10:1; about 9:1; about 8:1; about 7:1; about 6:1; about 5:1; about 4:1; about 3:1; about 2:1; about 1:2; about 3:2; about 5:2; about 1:3; about 2:3; about 4:3; about 5:3; about 1:4; about 3:4; about 5:4; about 1:5; about 2:5; about 3:5; about 4:5; about 2:1; about 2:3; about 2:5; about 3:1; about 3:2; about 3:4; about 3:5; about 4:1; about 4:3; about 4:5; about 5:1; about 5:2; about 5:3; and about 5:4. In a further embodiment, the copolymer is cross-linked with 1.4 butanediol diacrylate.

In another embodiment, the physiologically acceptable suspending agent is resorbable. In a further embodiment, the physiologically acceptable suspending agent is a buffered physiological solution. In another embodiment, the physiologically acceptable suspending agent comprises cross-linked sodium hyaluronate, non cross-linked sodium hyaluronate, collagen, and combinations thereof. In a further embodiment, the collagen is derived from natural sources or is synthetic.

The foregoing and other objects and aspects of the present invention are explained in greater detail in reference to the drawing and description set forth herein.

### BRIEF DESCRIPTION OF THE DRAWINGS

FIG. 1 depicts the location of the three fat pads in the human foot (e.g., under the heel, on the metatarsal head, and on the outer arch contact area).

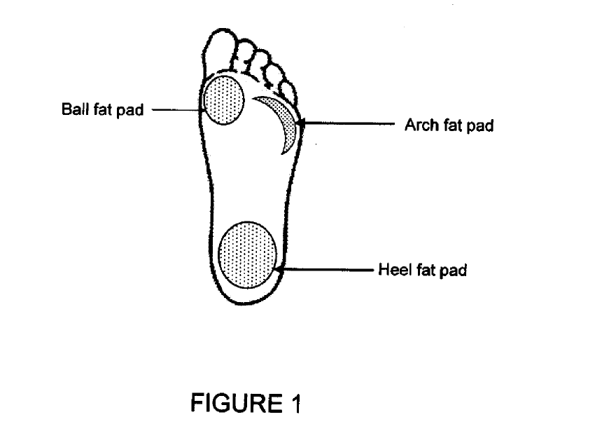

### DETAILED DESCRIPTION OF THE INVENTION

The foregoing and other aspects of the present invention will now be described in more detail with respect to embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a,” “an” and “the” are intended to include the plural forms as well, unless the context clearly indicates otherwise. Also, as used herein, “and/or” refers to and encompasses any and all possible combinations of one or more of the associated listed items. Furthermore, “about,” as used herein when referring to a measurable value such as an amount of a compound, dose, time, temperature is meant to encompass variations of 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

“Administration” as used herein, refers broadly to any means by which a composition is given to a patient. A preferred route of administration is by injection and unless otherwise indicated, any reference herein to “administration” includes “administration by injection.”

“Atrophic fat pad,” as used herein, refers broadly to a fat pad that has underwent a reduction in volume, weight, thickness, or size. This term may be used to describe the condition of the fat pad in an individual suffering from “fat pad atrophy.”

“Autologous solution,” as used herein, refers broadly to any liquid that is autologous to a patient to be treated, or that originates from the patient to be treated.

“Biocompatible,” as used herein, refers broadly to the quality of a solution that can be compatible with biological tissues, that is not toxic to biological tissues, and that is tolerated by the biological tissues.

“Biocompatible polymer,” as used herein refers broadly to a polymer that is substantially non-toxic and does not tend to produce substantial immune responses, clotting, or other undesirable effects.

“Composition” as used herein, refers broadly to any composition containing an agent or agents. The composition may comprise a dry formulation, an aqueous solution, a paste formulation, an organic solution formulation, a gel formulation, a jelly formulation, or a sterile composition. Compositions comprising the molecules described herein can be stored in freeze-dried form and can be associated with a stabilizing agent such as a carbohydrate. Composition, as used herein, includes “pharmaceutical compositions” which refers broadly to a chemical or biological composition suitable for administration to a subject (e.g., mammal). Such compositions may be specifically formulated for administration via one or more of a number of routes, including but not limited to cutaneous, epicutaneous, infusion, intradermal, intrathecal, subcutaneous, subdermal, and transdermal. In addition, the composition may be in the form of a capsule, drops, foams, gel, gum, injection, liquid, patch, pill, porous pouch, powder, or tablet.

“Copolymer,” as used herein, refers broadly to a polymer comprising more than one different monomer in the polymer chain (e.g., heteropolymer). The constituent monomers may be arranged in various ways, including alternating copolymers, periodic copolymers, statistical copolymers, random copolymers, graft copolymers, block copolymers, stereoblock copolymers, and other arrangements known in the art. Copolymers may be linear or branched, which includes star copolymers, brush copolymers, and comb copolymers, and other branched copolymer structures that are known in the art. For example, the molar ratio between the substituent monomers in a copolymer may be about 1:1000, 1:100, 1:10, 1:9, 1:8, 1:7, 1:6, 1:5, 1:4, 1:3, 1:2, or 1:1.

“Cushioning,” as used herein, refers broadly to the capacity of a fat pad to dissipate impact energy.

“Effective amount” or “effective,” as used herein, refers broadly to a dose that causes a relief of symptoms of a disease or disorder as noted through clinical testing and evaluation, or patient observation. “Effective amount” or “effective” further can further designate a dose that causes a detectable change in biological or chemical activity. The detectable changes may be detected and/or further quantified by one skilled in the art for the relevant mechanism or process. Moreover, “effective amount” or “effective” may designate an amount that maintains a desired physiological state, i.e., reduces or prevents significant decline and/or promotes improvement in the condition of interest (e.g., relief of symptoms). As is generally understood in the art, the dosage will vary depending on the administration routes, symptoms, age, and body weight of the patient but also depending upon the composition being administered.

“Fat pads,” as used herein, refers broadly to any cushions made of communicating pockets of fascia and filled with fatty acids that are present in mammals (e.g., humans).

“Forefoot,” as used herein, refers broadly to the foot portion about the ball and toes of the foot (i.e., the area substantially containing the phalanges, sesamoids, and the distal heads of the metatarsals.)

“Homopolymer,” as used herein, refers broadly to a polymer constructed of the same monomers.

“Increased” or “increase” as used herein, refers broadly to a quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater. In particular, the term “increase,” as used herein, refers broadly to make greater, as in number, size, strength, or quality; add to; and/or augment. “Increase,” as used herein, also encompasses expand, extend, prolong, augment, and/or enlarge. “Increase,” as used herein, additionally encompasses where a given parameter (e.g., level, amount, size, scope, duration, weight) is greater, as in number, size, strength, or quality, than it once was. Furthermore, the “increase” in any number, size, strength, or quality of a given parameter may be determined as between to two or more time points, especially if before or after a treatment, event, or administration of an agent or composition. Further, “increase” refers broadly to significant or detectable, functionally, analytically, and/or clinically, changes in the number, size, strength, or quality of a given parameter in question.

“Mammal” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to humans, non-human primates, alpacas, camels, capybaras, cats, chimpanzees, chinchillas, cows, dogs, goats, gorillas, horses, llamas, mice, pigs, rats, sheep, and tapirs. Mammals include but are not limited to bovine, canine, equine, feline, murine, ovine, porcine, primate, and rodent species. Mammal also includes but is not limited to those listed on the Mammal Species of the World maintained by the National Museum of Natural History, Smithsonian Institution in Washington, D.C.

“Midfoot,” as used herein, refers broadly to the foot portion lying between the forefoot and rearfoot as defined above. The midfoot portion therefore lies rearwardly of the distal heads of the metatarsals and forwardly of the calcaneus and talus, and substantially contains the cuboid, navicular, cuneiforms, and includes the base and a substantial portion of the shaft of the metatarsals.

“Patient” as used herein, refers broadly to any animal who is in need of treatment either to alleviate a disease state or to prevent the occurrence or reoccurrence of a disease state. Also, “Patient” as used herein, refers broadly to any animal who has a history of disease, symptoms, signs, was previously diagnosed, or is a member of a patient population for a disease. The patient may be a clinical patient such as a human or a veterinary patient such as a companion, domesticated, livestock, exotic, or zoo animal.

“Physiologically acceptable suspending agent” or “pharmaceutically acceptable suspending agent,” as used herein, refers broadly to physiologically inert substances (e.g., solvent or dispersion media) that acts to reduce the sedimentation rate of particles (e.g., pliable biocompatible material) in suspension or increases the viscosity of the suspension. Physiologically acceptable suspending agent includes aqueous solutions, dispersions and polymers. The use of such media and agents for physiologically acceptable suspending agents is well known in the art.

“Rearfoot,” as used herein, refers broadly to the area about the heal portion of the foot substantially containing the heel bones (i.e., the calcaneus and talus.)

“Restoring,” as used herein, refers broadly to the action of bringing back, totally or partly, specific normal physiological properties such as the physico-chemical, the physical (e.g., thickness), the mechanical (e.g., absorbing) or the physiological functions (e.g., cushioning).

“Self-gelling,” as used herein, refers broadly to the ability of turning into gels under specific conditions such as the internal composition or/and the action of external stimuli. Self-gelling encompasses pH-triggered or pH-controlled gelling, thermo-gelling, and ionic gelling.

“Signs” of disease, as used herein, refers broadly to any abnormality indicative of disease, discoverable on examination of the patient; an objective indication of disease, in contrast to a symptom, which is a subjective indication of disease.

“Solution,” as used herein, refers broadly to any liquid, organic or aqueous, low to high viscosity systems, to any dispersions of solids into liquid, organic or aqueous, low to high viscosity systems, and to any gelled, organic or aqueous, extrudable or injectable, systems. Such solutions may comprise soluble small-size molecules, soluble monomers, soluble oligomers, soluble polymers and copolymers as well as nonsoluble solid organic or mineral entities such as microparticles or nanoparticles.

“Subjects” as used herein, refers broadly to anyone suitable to be treated according to the present invention include, but are not limited to and mammalian subjects (e.g., humans). The present invention can also be carried out on animal subjects, particularly mammalian subjects such as mice, rats, dogs, cats, cattle, goats, sheep, and horses for veterinary purposes, and for screening, testing, and development purposes. “Subjects” is used interchangeably with “patients.”

“Swellable” microspheres, as used in the present invention, refers to microspheres that are capable of being enlarged in size, yet still retain substantially the same shape, upon certain conditions such as contacting physiological fluids.

“Symptoms” of disease as used herein, refers broadly to any morbid phenomenon or departure from the normal in structure, function, or sensation, experienced by the patient and indicative of disease.

“Therapy” or “therapeutic” as used herein refers broadly to treating a disease, arresting, or reducing the development of the disease or its clinical symptoms, and/or relieving the disease, causing regression of the disease or its clinical symptoms. Therapy encompasses treatment, cure, remedy, minimization, reduction, alleviation, and/or providing relief from a disease, signs, and/or symptoms. Therapy encompasses an alleviation of signs and/or symptoms in patients with ongoing disease signs and/or symptoms (e.g., pain, inflammation.) The term “reduced,” for purpose of therapy, refers broadly to the clinical significant reduction in signs and/or symptoms. Therapy includes treating chronic disease (“maintenance”) and acute disease.

“Treating” or “treatment,” as used herein, refers broadly to a course of therapy where signs and/or symptoms are present in the patient. The term “reduced,” for purpose of therapy, refers broadly to clinically significant reduction in signs and/or symptoms. Treatment can alleviate, decrease, lessen, relieve, remedy, repair, and/or soothe a disease, signs, and/or symptoms. By the terms “treating” or “treatment” of a condition, it is intended that the severity of the disorder or the symptoms of the condition are reduced, or the condition is partially or entirely eliminated, as compared to that which would occur in the absence of treatment. Treatment does not require the achievement of a complete cure of the condition. Treatment includes treating chronic disease (“maintenance”) and acute disease.

### Podiatric Filler Compositions

Podiatric filler compositions may comprise a pliable biocompatible material and a physiologically acceptable suspending agent. The pliable biocompatible material may be a polymer (including copolymers), calcium hydroxylapatite, or autologous fat, and the physiologically acceptable suspending agent may be any solvent, suspension, colloid, gel, foam, macromolecules, polymers (including copolymers), or solution. Supplementary active compounds may be incorporated into the podiatric filler compositions (e.g., analgesics, anti-inflammatory agents).

Polymers acceptable for use in the present invention include but are not limited to alginate, cellulose (including substituted cellulose), chitosan, chondrotin sulfate, fatty acids, gelatin, glycol, glycosaminoglycans, hyaluronic acid, polyacrylamide, polyethylene glycol, polylysine, polypeptides, polysaccharides, proteoglycans, and silicone.

The pliable biocompatible material may not be not absorbable or degradable by the body and is inert. The podiatric filler composition may be pliable because it is a characteristic that is important for podiatric applications. The material may also be hydrophobic because it is beneficial for expansion of the podiatric filler. The podiatric filler composition may comprise a colorant agent (e.g., dye, contrast agent, tint) for easier administration, monitoring, or removal. The podiatric filler composition may produce little or no immunological response in the host tissue or no immunological response in the host tissue. For example, the administration of the podiatric filler composition may not lead to the formation of inflammatory granuloma and scarring.

The materials described herein may be purified from cells that naturally express it, purified from cells (e.g., bacteria, plant, insect, mammal) that have been altered to express it (e.g., recombinant), or synthesized using standard methods known in the art. The pliable biocompatible materials described herein may also be obtained from commercial sources as a purified or isolated form (e.g., natural, recombinant, or synthetic sources).

The pliable biocompatible material solid particles may be a dust, powder, non-porous microspheres, nanoparticles, granules, grains, or microbeads. The solid particles may easily administered (e.g., injected), remain immobilised at the administration site, and be tolerated by the tissue (e.g., little or no side effects). Generally, the solid particles may be small enough to be injected through a cannula of an injection syringe to the desired site and not be identified by human touch as a single foreign body under the skin. The pliable biocompatible material may be a solid particle with a diameter such that it is not washed away through lymph tracts or other tissue tracts from the site to which they have administered.

The pliable biocompatible material solid particles having a spherical form or a spherical like form. Spherical formed particles have an advantage in that they form a closely packed arrangement at the site where they are administered.

The solid particles may have an average diameter of about 0.01, 0.02, 0.03, 0.04, 0.05, 0.06, 0.07, 0.08, 0.09, or 0.10 μm, as well as at least about 0.1, 0.2, 0.3, 0.4, 0.5, 0.6, 0.7, 0.8, 0.9, or 1.0 μm, as well as at least about 1, 2, 3, 4, 5, 6, 7, 8, 9, or 10 μm, as well as at least about 10, 11, 12, 13, 14, 15, 16, 17, 18, 19, or 20 μm, as well as at least about 10, 20, 25, 30, 35, 40, 45, 50, 55, 60, 65, 70, 75, 80, 85, 90, 95, 100, 125, 150, 175, or 200 μm, and all increments therein. Preferably, the diameter of the particle may about 0.05-5, 0.01-10, 1-5, 4-5, 1-10, 5-10, 2-50, 15-60, 15-60, or 10-100 μm.

The solid particles may be swellable microspheres which are highly water absorbing microspheres capable of swelling to many times of their original sizes under certain conditions. Swellable microspheres are capable of swelling upon contacting with medium resembling the properties of physiological fluids, thus allowing the microspheres to secure themselves into position after injection into the body. Furthermore, the microspheres are substantially spherical and can be easily calibrated so that their sizes can be accurately determined. The microspheres of the invention have diameters from about 10-100, 10-120, 10-200, 10-300, 10-400 μm before swelling. After injection and swelling, the microspheres have average diameters larger than about 10, 20, 30, 40, 50, 60, 70, 80, 90, or 100 μm. See U.S. Pat. No. 6,436,424.

The swellable microspheres can be enlarged to about 4 times of their original diameter or 15 times of their original volume. The degree of swelling can be controlled by controlling factors such as the solvents in which they are suspended, specific polymers used to make the microspheres and degree of crosslinking. This property enables the microspheres to be easily injected through needles of 30 gauge or smaller, yet be enlarged and secured at the injection site and of sufficient size to avoid or reduce the chance of being eliminated by the immune system of the mammal.

The podiatric filler composition may be administered in a volume of at least about 0.01, 0.02, 0.03, 0.04, 0.05, 0.06, 0.07, 0.08, 0.09, or 0.10 μL, as well as at least about 0.1, 0.2, 0.3, 0.4, 0.5, 0.6, 0.7, 0.8, 0.9, or 1.0 μL, as well as at least about 1, 2, 3, 4, 5, 6, 7, 8, 9, or 10 μL.

The podiatric filler composition may be administered in a volume of at least about 10, 20, 30, 40, 50, 60, 70, 80, 90, or 100μL. The volume of the composition administered to the patient may be about 1-5, 5-10, or 1-100 μL.

The podiatric filler composition may be administered in a volume of at least about 0.01, 0.02, 0.03, 0.04, 0.05, 0.06, 0.07, 0.08, 0.09, or 0.10 mL (cc), as well as at least about 0.1, 0.2, 0.3, 0.4, 0.5, 0.6, 0.7, 0.8, 0.9, or 1.0 mL, as well as at least about 1, 2, 3, 4, 5, 6, 7, 8, 9, or mL (cc). The volume of the composition administered to the patient may be about 1-5, 5-10, or 1-10 mL (cc).

The podiatric filler composition may comprise a physiological acceptable suspending agent of at least about 0.01, 0.02, 0.03, 0.04, 0.05, 0.06, 0.07, 0.08, 0.09, or 0.10% (w/w), as well as at least about 0.1, 0.2, 0.3, 0.4, 0.5, 0.6, 0.7, 0.8, 0.9, or 1.0% (w/w), as well as at least about 1, 2, 3, 4, 5, 6, 7, 8, 9, or 10% (w/w). The podiatric filler may comprise a physiological acceptable suspending agent of at least about 1, 5, 10, 15, 20, 25, 30, 35, 40, 45, 50, 60, 65, 70, 75, 80, 85, 90, 95, 96, 97, 98, 99, or 99.5% (w/w).

The podiatric filler composition may comprise a pliable biocompatible material of at least about 0.01, 0.02, 0.03, 0.04, 0.05, 0.06, 0.07, 0.08, 0.09, or 0.10% (w/w), as well as at least about 0.1, 0.2, 0.3, 0.4, 0.5, 0.6, 0.7, 0.8, 0.9, or 1.0% (w/w), as well as at least about 1, 2, 3, 4, 5, 6, 7, 8, 9, or 10% (w/w). The podiatric filler may comprise a pliable biocompatible material of at least about 1, 5, 10, 15, 20, 25, 30, 35, 40, 45, 50, 60, 65, 70, 75, 80, 85, 90, 95, 96, 97, 98, 99, or 99.5% (w/w).

The podiatric filler composition may comprise a physiological acceptable suspending agent of at least about 0.01, 0.02, 0.03, 0.04, 0.05, 0.06, 0.07, 0.08, 0.09, or 0.10 μg/mL, as well as at least about 0.1, 0.2, 0.3, 0.4, 0.5, 0.6, 0.7, 0.8, 0.9, or 1.0 μg/mL, as well as at least about 1, 2, 3, 4, 5, 6, 7, 8, 9, or 10 μg/mL. The podiatric filler may comprise a physiological acceptable suspending agent at least about 1, 2, 3, 4, 5, 6, 7, 8, 9, 10, 15, 20, 25, 30, 35, 40, 45, 50, 60, 65, 70, 75, 80, 85, 90, 95, 96, 97, 98, 99, 99.5, or 100 μg/mL.

The podiatric filler composition may comprise about a physiological acceptable suspending agent of at least about 0.01, 0.02, 0.03, 0.04, 0.05, 0.06, 0.07, 0.08, 0.09, or 0.10 mg/mL, as well as at least about 0.1, 0.2, 0.3, 0.4, 0.5, 0.6, 0.7, 0.8, 0.9, or 1.0 mg/mL, as well as at least about 1, 2, 3, 4, 5, 6, 7, 8, 9, or 10 mg/mL. The podiatric filler may comprise a physiological acceptable suspending agent of at least about 1, 2, 3, 4, 5, 6, 7, 8, 9, 10, 15, 20, 25, 30, 35, 40, 45, 50, 60, 65, 70, 75, 80, 85, 90, 95, 96, 97, 98, 99, 99.5, or 100 mg/mL.

The podiatric filler composition may comprise a pliable biocompatible material of at least about 0.01, 0.02, 0.03, 0.04, 0.05, 0.06, 0.07, 0.08, 0.09, or 0.10 μg/mL, as well as at least about 0.1, 0.2, 0.3, 0.4, 0.5, 0.6, 0.7, 0.8, 0.9, or 1.0 μg/mL, as well as at least about 1, 2, 3, 4, 5, 6, 7, 8, 9, or 10 μg/mL. The podiatric filler may comprise a pliable biocompatible material of at least about 1, 2, 3, 4, 5, 6, 7, 8, 9, 10, 15, 20, 25, 30, 35, 40, 45, 50, 60, 65, 70, 75, 80, 85, 90, 95, 96, 97, 98, 99, 99.5, or 100 μg/mL.

The podiatric filler composition may comprise a pliable biocompatible material of at least about 0.01, 0.02, 0.03, 0.04, 0.05, 0.06, 0.07, 0.08, 0.09, or 0.10 mg/mL, as well as at least about 0.1, 0.2, 0.3, 0.4, 0.5, 0.6, 0.7, 0.8, 0.9, or 1.0 mg/mL, as well as at least about 1, 2, 3, 4, 5, 6, 7, 8, 9, or 10 mg/mL. The podiatric filler may comprise a pliable biocompatible material of at least about 1, 2, 3, 4, 5, 6, 7, 8, 9, 10, 15, 20, 25, 30, 35, 40, 45, 50, 60, 65, 70, 75, 80, 85, 90, 95, 96, 97, 98, 99, 99.5, or 100 mg/mL.

The podiatric filler composition may comprise at least 1, 2, 3, 4, 5, 6, 7, 8, 9, or 10 different pliable biocompatible materials. The podiatric filler composition may comprise at least 1, 2, 3, 4, 5, 6, 7, 8, 9, or 10 different physiological acceptable suspending agents. For example, the podiatric filler composition may comprise at least one pliable biocompatible material and at least one physiological acceptable suspending agent. Further, the physiological acceptable suspending agent may comprise a mixture of different components (e.g., mixture of fatty acids).

The podiatric filler composition may be under gel (or solid form) at low to room temperature, e.g., about 20° C. and below, but may become more or less a viscous liquid at higher temperatures, e.g., about above 35-40° C.

The podiatric filler composition may become highly viscous or turn into a gel after being injected into the fat pad by one of the following processes: (a) Gelling: the solution is injected as a liquid, and later turns in situ into a gel, within the fat pad; (b) Polymerisation in situ: the solution containing monomers and/or oligomers, or a mixture of two or more different monomers, is injected as a liquid, and later polymerises or co-polymerises in situ, within the fat pad; (c) Concentration: the solution contains a viscous component mixed with an appropriate metabolically absorbable liquid vehicle, to reduce viscosity and to allow injectability, the solvent or vehicle, after injection in the pad, being absorbed by the patient, thus increasing the concentration and hence the viscosity of the viscous component.

The viscosity (η) may be determined using methods known in the art (e.g., a rotating viscometer at 20° C., Ostwald viscometer). See, e.g., Akhare, et al. (2007) Indian Journal of Pure & Applied Physics 45: 984-986. The viscosity of the podiatric filler composition may be at least about 100, 200, 300, 400, 500, 600, 700, 800, 900, or 1,000 mPa·S. (millipascal-second).

### Acrylate Copolymers

A copolymer suitable for use in the present invention comprises an unsubstituted acrylate monomer and a substituted acrylate monomer (e.g., acrylate/methacrylate copolymer). The substituted acrylate monomer may be substituted with a methyl group. Other alkyl group derivatives, e.g., one, two, three, four, five, or six carbons in length (e.g., methyl, ethyl, propyl, isopropyl, butyl, tert-butyl, pentyl, hexyl) may be used as a substituent in the substituted acrylate monomer. Cyclic alkyl groups with four, five, or six carbons may be the substituent group on the substituted acrylate monomer. The substituent group for the substituted acrylate monomer may a halogen group. For example, the halogen group may be flourine (F), chlorine (Cl), bromine (Br), iodine (I), or astatine (At). Additionally, a nitrile group is another possible substituent for the substituted acrylate monomer. An acrylate/methacrylate copolymer that may be used is the copolymer used in AcryS of MA60BM intraocular lenses (Alcon Laboratories).

The inventor surprisingly found that a copolymer comprises an unsubstituted acrylate monomer and a substituted acrylate monomer (e.g., acrylate/methacrylate copolymer) in a finely ground powder form is highly desirable copolymer for use as a pliable biocompatible material because of the lack of immunogenicity. For example, when introduced into human tissue a copolymer comprises an unsubstituted acrylate monomer and a substituted acrylate monomer (e.g., acrylate/methacrylate copolymer) has few or no side-effects. Further, tissue a copolymer comprises an unsubstituted acrylate monomer and a substituted acrylate monomer (e.g., acrylate/methacrylate copolymer) tends not form granulomas when introduced into human tissue, in contrast with other polymers used in medicine.

### Calcium Hydroxylapatite

Calcium hydroxylapatite may be used as a pliable biocompatible material. Calcium hydroxylapatite is a component of bone and teeth may be used in the form of microspheres suspended in an aqueous carrier (e.g., RADIESSE® (calcium hydroxylaptite)). The host tissue reacts to the injected calcium hydroxyapatite microspheres to stimulate collagen production to encapsulate the microspheres. Calcium hydroxylapatite may be admixed with a polymer to form a pliable composite.

### Collagen

Collagen may be employed as a pliable biocompatible material. Collagen is the major insoluble fibrous protein in the extracellular matrix and in connective tissue. Collagen that may be used including but not limited to autologous, synthetic, recombinant, and natural collagen. See U.S. Pat. Nos. 4,544,516; 4,698,360; and 5,856,308 and WO 2006/098326. Collagen may be isolated from cattle, pigs, or humans (e.g., autologous). Collagen may also be made by recombinant means including transgenic animals, bacteria, insect cells, and transgenic plants. See U.S. Pat. Nos. 6,111,165; 6,413,742; and 7,238,783; U.S. Patent Application Publication No. 2009/0030184. For example, Autologen® (collagen) is a collagen extracted from a patient, sterilized and processed into injectable form. Alternatively, Isolagen® (pharmaceutical composition of fibroblasts) a preparation of live cloned fibroblasts, such as collagen-producing cells, which are also derived from a patient's own podiatric and prepared into liquid form may be used to produce collagen in situ. Collagen may be cross-linked with glutaraldehyde. Further examples of commercially available collagen include but are not limited to bovine, human, cross-linked porcine [EVOLENCE®]) and synthetic (FG-5030 synthetic human collagen (type III)) COSMOPLAST® (highly purified human collagen), COSMODERM® (highly purified human collagen).

### Fatty Acids

At least one fatty acid may be used as a pliable biocompatible material. Fatty acids suitable for use in this invention include but are not limited to natural or unnatural saturated and mono- or poly-unsaturated fatty acids. Additionally, exemplary fatty acids include but are not limited to palmitate, stearate, myristate, palmitoleate, oleate, vaccenate, linoleate, and their acyclic, cyclic, heterocyclic, aromatic ester derivatives thereof. The fatty acid derivatives may comprise one or more groups including but not limited to hydroxy, acyloxy, aryloxy, amino, sulfhydryl, sulfonate, sulfate, phosphonate, phosphate, bis-, tris- and poly-phosphonates and phosphates, phosphatidyl, nucleosides, oligosaccharides, polysaccharides, polyols, and a mixture thereof. An exemplary fatty acid mixture comprises oleoate and palmitate (e.g., 17% palmitic acid and 83% oleic acid (w/w)). Another exemplary fatty acid mixture comprise myristate, palmitate, stearate, palmitoleate, vaccenate, oleate, and linoleate (e.g., myristate 1.9%, palmitate 15.9%, stearate 1.7%, palmitoleate 12.3%, vaccenate 4.8%, oleate 46.4% and linoleate 17.0% (w/w)).

### Hyaluronic Acid

Hyaluronan (hyaluronic acid or hyaluronate) may be used as a pliable biocompatible material. Hyaluronan is an anionic, nonsulfated glycosaminoglycan distributed widely throughout connective, epithelial, and neural tissues. Exemplary hyaluronan compositions include but are not limited to sodium hyaluronate, JUVEDEM® (cross-linked hyaluronic acid), RESTYLANE® (hyaluronic acid), and PERLANE® (hyaluronic acid). Hyaluronan made be admixed with a another agent such as an analgesic (e.g., PREVELLE® SILK (hyaluronic acid with lidocaine)).

### Polyacrylates

Polyacrylate may be used as a pliable biocompatible material. Polyacrylates that may be used include but are not limited to polymethylacrylate and polymethylmethacrylate (PMMA). ARTEFILL® (polymethylmethacrylate (PMMA) microspheres) is a permanent microsphere-based injectable filler comprising non-resorbable microspheres having a diameter of between 30-42 μm and a smooth surface. Treatment with PMMA is considered permanent, not only because of the volume increase under the defect, but also because of the presumed life-long stimulation of collagen deposition beneath the defect. The electrical charge of filler microbeads appears to play a role in attracting and activating macrophages, which promotes formation of foreign body giant cells, then fibroblasts, and thereby increases the composition of new connective tissue. Adverse side-effects may include lumpiness and inflammatory granulomas. PMMA may be admixed with another filler for use as a podiatric filler composition. For example, collagen, may be combined with PMMA in a ratio of 20% PMMA to 80% collagen. This collagen/PMMA mixture may further comprise an anesthetic, such as lidocaine (e.g., 3% lidocaine anesthetic solution). See, e.g., U.S. Pat. No. 5,344,452.

### Polyacrylamide

Polyacrylamide (IUPAC poly(2-propenamide) or poly(1-carbamoylethylene)) is a polymer (—CH₂CHCONH₂—) formed from acrylamide. Polyacrylamide may be admixed with another compound to form a composite. For example, AQUAMID® (hydrophilic polyacrylamide) is a composition currently used in tissue augmentation. See U.S. Patent Application Publication No. 2006/0115457 and 2008/0292706.

### Polyalkylene Glycol

Polyalkylene glycol polymers may be used as a pliable biocompatible material. Polyalkylene glycol polymers include but are not limited to straight or branched polyalkylene glycol polymers such as polyethylene glycol, polypropylene glycol, and polybutylene glycol, and further includes the monoalkylether of the polyalkylene glycol. The polyalkylene glycol polymer may be a lower alkyl polyalkylene glycol moiety such as a polyethylene glycol moiety (PEG), a polypropylene glycol moiety, or a polybutylene glycol moiety. PEG has the formula —HO(CH₂CH₂O)ₙH, where n can range from about 1-100, 5-30, or 1-4000. The PEG moiety can be linear or branched. PEG may be attached to groups such as hydroxyl, alkyl, aryl, acyl, or ester. For example, PEG may be an alkoxy PEG, such as methoxy-PEG (or mPEG), where one terminus is a relatively inert alkoxy group, while the other terminus is a hydroxyl group. Further polyalkylene glycol polymers include but are not limited to poly(ethylene glycol), poly(propylene glycol), and its copolymers, poly(ethylene glycol) copolymers with other synthetics such as poly(hydroxy acids), poly(vinyl alcohol), poly(vinyl pyrrolidone), and mixture thereof.

### Polylactic Acid

Polylactic acid microspheres may be used as a pliable biocompatible material. The microspheres may have a diameter of about 2-50 μm. The polylactic acid microspheres become porous after a first phase of moderate inflammation in the host tissue, followed by a second stronger inflammation in which foreign body giant cells phagocytose the microspheres and accelerate the implant's degradation. The improvement of defects is immediate and has been reported to last up to two years. The non-resorbable microspheres add permanent volume under the treated defect, as well as stimulate the host to produce collagen fibers around the implant. An example of polylactic acid which may be used is SCULPTRA® (injectable polylactic acid).

### Proteoglycans

Proteoglycans may be used as a pliable biocompatible material. Proteoglycans are glycoproteins in connective tissue comprising a core protein and one or more covalently attached glycosaminoglycan (GAG) chain(s). Proteoglycans can be categorized depending upon the nature of their glycosaminoglycan chains and include but are not limited to chondroitin sulfate, dermatan sulfate, heparin, heparan sulfate, and keratan sulfate.

### Cross-Linking Agents

The pliable biocompatible materials (including polymers) described herein may be cross-linked using 1.4 butanediol diacrylate. Exemplary cross-linking agents may be any terminally ethylenically unsaturated compound having more than one unsaturated group (i.e., a multiplicity of unsaturated groups.) See U.S. Pat. No. 5,741,923. Other exemplary cross-linking agents include, but are not limited to ethylene glycol diacrylate or dimethacrylate, diethylene glycol diacrylate or dimethacrylate, triethylene glycol diacrylate or dimethacrylate, tetraethylene glycol diacrylate or dimethacrylate, polyethylene glycol diacrylate or dimethacrylate, trimethylolpropane triacrylate or trimethacrylate, bisphenol A diacrylate or dimethacrylate, ethoxylated bisphenol A diacrylate or dimethacrylate, pentaerythritol tri- and tetra-acrylate or methacrylate, tetramethylene diacrylate or dimethacrylate, methylene bisacrylamide or methacrylamide, dimethylene bisacrylamide or methacrylamide, N,N′-dihydroxyethylene bisacrylamide or methacrylamide, hexamethylene bisacrylamide or methacrylamide, decamethylene bisacrylamide or methacrylamide, divinyl benzene, vinyl methacrylate, and allyl methacrylate. Additional exemplary cross-linking agents include 1,3-bis(4-methacryloyl oxyalkyl)tetra disiloxane and similar poly(organo-siloxane) monomers. See U.S. Pat. No. 4,153,641. Another group of exemplary cross-linking agents are the resonance-free di(alkylene tertiary amine) cyclic compounds (e.g., N,N′-divinyl ethylene urea). See U.S. Pat. No. 4,436,887. Further exemplary cross-linking agents include di- or polyvinyl ethers of di- or polyvalent alcohols such as ethylene glycol divinyl ether.

### Physiologically Acceptable Suspending Agent

The physiologically acceptable suspending agent generally acts to the reduce viscosity, reduce the sedimentation rate of particles in suspension, and/or allow injectability at room temperature. When admixed with a pliable biocompatible material it forms a composition that may be a suspension, colloid, gel, or solution. The physiologically acceptable suspending agent may be resorbable.

The physiologically acceptable suspending agent may be a solution comprising an aqueous liquid or a non-aqueous liquid such as water-soluble and water-insoluble solvents or liquid chemicals (e.g., alcoholic solvents, alkylene glycols (ethylene glycol), dimethyl sulfoxide, ethanol, ethyl acetate, ethylene glycol, glycerol, isopropyl alcohol, N-methyl-pyrrolidone, physiological buffers, poly-alcohols, propylene carbonate, triacetin, water, and mixtures thereof).

The physiologically acceptable suspending agent may be a buffered physiological solution (e.g., normal saline, Ringer's solution, phosphate buffered saline (PBS), TRIS-buffered saline (TBS), HEPES-buffered saline (HBS)). The buffered physiological solution may have a pH or about 7.0 to about 8.0 including a pH of about 7.1, 7.2, 7.3, 7.4, 7.5, 7.6, 7.7, 7.8, or 7.9.

The physiologically acceptable suspending agent may be a polymer including copolymers as described herein. For example, hyaluronan, collagen, or fatty acids may used as physiological acceptable suspending agents.

The physiologically acceptable suspending agent may be stored in one form (e.g., gel) and then prepared (e.g., heated) to change into an injectable form (e.g., solution) prior to administration. For example, the solution may be stored as a gel at a temperature below the physiological temperature and heated above the physiological temperature prior to use in order for the solution to be injectable. The suspending agents may contain a tenside (surfactant), for instance Tween 80® (polyethoxysorbitanoleate) because surfactants change the surface tension of water so that the solid particles and in particular the polymer particles float better.

The solution may be a self-gelling solution such as a stimuli-triggered self-gelling polymeric solution, and preferably a thermo-gelling solution. This thermo-gelling solution may be a thermo-gelling chitosan-based aqueous. WO 99/07416. Self-gelling solutions may be liquid at low to room temperature (e.g., about 25° C. or below) and may form a solid gel at a higher temperature, e.g., above 30° C. Inversely, such self-gelling solutions may be liquid at high temperatures, e.g., above 40° C., but may form a gel at a lower temperature, e.g., below 40° C. Typical thermo-gelling polymeric solutions may be designed with polymers selected among poly(acrylic acid), methyl cellulose, hydroxyethyl cellulose, hydroxypropyl methyl cellulose, poly(ethylene oxide)-based triblock copolymers, or chitosan. Other injectable self-forming systems, e.g., Lower critical solution temperature (LCST), liquid crystalline, polymer precipitation (solid), precipitation in situ, coagulation may also be used.

### Pharmaceutical Compositions

The preferred forms of administration in the present invention are injectable forms known in the art of pharmaceutics. The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, and pharmaceutically compatible carriers.

For each of the recited embodiments, the composition can be administered by a variety of dosage forms as known in the art. Any biologically-acceptable dosage form known to persons of ordinary skill in the art, and combinations thereof, are contemplated. Examples of such dosage forms include, without limitation, elixirs, emulsions, liquids, solutions, suspensions, syrups, emulsions, beads, powders, gum, granules, particles, microparticles, dispersible granules, topicals, patches, implants, depot implants, injectables, infusions, and combinations thereof.

Moreover, the compounds (e.g., analgesics, anti-inflammatory agents) described herein can be formulated in a time release formulation, for example in a composition that includes a slow release polymer. The podiatric filler may be prepared with carriers that will protect the compound against rapid release, such as a controlled release formulation, including implants and microencapsulated delivery systems. Biodegradable, biocompatible polymers may be used (e.g., ethylene vinyl acetate, polyanhydrides, polyglycolic acid, collagen, polyorthoesters, polylactic acid and polylactic, and polyglycolic copolymers (PLG)). Many methods for the preparation of such formulations are known to those skilled in the art.

### Analgesics

The podiatric filler composition may be administered in conjunction with an analgesic. The analgesic may be included in the podiatric filler composition, or administered sequentially, concurrently, or subsequently with the administration of the podiatric filler composition. Analgesics that may be used in the present invention include but are not limited to acetaminophen (Tylenol®), amitriptyline, carbamazepine, codeine (Tylenol #2,3,4®), dihydromorphine, fentanyl patch (Duragesic®), Flupirtine, fluriprofen, gabapentin, hydrocodone APAP (Vicodin®), hydromorphone (Palladone®), ibuprofen, ketoprofen, morphine (MS Contin®), oxycodone (Percocet®, OxyContin®, Percodan®), pentazocine (Talwin NX®), pethidine, phenacetin, pregabalin, propoeylphene (Darvon®), propoyl APA (Darvocet®), salicylamide (aspirin), tramadol (Ultram®), tramadol APAP (Ultracet®), and voltaren.

### Local Anesthetic

The podiatric filler composition may be administered in conjunction with a local anesthetic. The local anesthetic may be included in the podiatric filler composition, or administered sequentially, concurrently, or subsequently with the administration of the podiatric filler composition. Local anesthetic that may be used in the present invention include but are not limited to amethocaine, articaine, benzocaine, bupivacaine, Carbocaine® (mepivacaine), cocaine, cinchocaine, chloroprocaine, cyclomethycaine, dibucaine, dimethocaine, EMLA® (eutectic mixture of lidocaine and prilocalne), etidocaine, larocaine, levobupivacaine, lidocaine, lignocaine, Novocaine® (procaine), piperocaine, Polocalne® (mepivacaine), prilocalne, proparacaine, propoxycaine, ropivacaine, saxitoxin, tetracaine, tetrodotoxin, and trimecaine.

### Anti-Inflammatory Agents

The podiatric filler composition may be administered in conjunction with an anti-inflammatory agent. The anti-inflammatory agent may be included in the podiatric filler composition, or administered sequentially, concurrently, or subsequently with the administration of the podiatric filler composition. Anti-inflammatory agents such as non-steroidal anti-inflammatory drugs (NSAIDs) are classified into groups based on their chemical structure: (1) propionic acid derivatives, (2) acetic acid derivatives, (3) enolic acid derivatives, (4) fenamic acid derivatives, and (5) selective COX-2 inhibitors. Any member of these classes may be used as an anti-inflammatory agent. Exemplary non-steroidal anti-inflammatory drugs (NSAIDs) that may be used in the present invention include but are not limited to acetaminophen (Tylenol®), azapropazone, diclofenac, diflunisal, etodolac, fenbufen, fenoprofen, flurbiprofen, ibuprofen (Motrin®), indomethacin (indometacin), ketoprofen, ketorolac, mefenamic, meloxicam, nabumetone, naproxen, phenylbutazone, piroxicam, salicylates (e.g., acetylsalicylic acid (Aspirin), amoxiprin, benorylate/benorilate, choline magnesium salicylate, Diflunisal®, ethenzamide, faislamine, methyl salicylate, magnesium salicylate, salicyl salicylate, and salicylamide), sulindac, tenoxicam, tiaprofenic acid, and tolfenamic acid.

### Methods of Manufacture

The podiatric filler composition may be produced by a method comprising combining the components described herein. For example, the method of manufacture may comprise admixing a pliable biocompatible material with a physiologically acceptable suspending agent to make a podiatric filler composition. The pliable biocompatible material may be admixed with a physiologically acceptable suspending agent prior to administration, including immediately prior to administration. For example, the pliable biocompatible material may be admixed with a physiologically acceptable suspending agent in a mixing vessel (or even the syringe) and then immediately administered to the patient.

The composition may be calibrated to approximate the functional properties of the mixture of fatty acids normally present in human fat pads. For example, the relative proportion of a pliable biocompatible material and the physiological acceptable suspending agent may be determined to achieve mechanical properties similar to those of the natural fatty acid composition of the fat pad.

The physiological acceptable suspending agent may be added to a pliable biocompatible material until a desired thickness, viscosity, or consistency is reached. The mixing ratio of the components of the suspending agent may be chosen according to the needs and in particular according to the size of the syringe used for the injection. The solid particles used according to the present invention, the particles may be suspended or slurried in a physiological acceptable suspending agent.

### Methods of Treatment

The present invention provides a variety techniques for ameliorating the effects of fat pad atrophy comprising administration of an effective amount of a podiatric filler composition. It is intended that the method described herein can be applied similarly to any other fat pads of human and mammalian bodies, such fat pads being defined as being closed cushions of communicating chambers filled with fatty acids, for restoring totally or partly the physical functions of atrophic, damaged, deteriorated, or degenerated pads with an injectable solution, by injecting in the pad and restoring first the thickness of the natural pad for a long enough time, from a few weeks to permanence.

The present invention provides a method for restoring a fat pad comprising administration of an effective amount of a podiatric filler composition. The compositions described herein may be used in a method of augmenting soft tissue, including foot fat pads, comprising administration of a podiatric filler composition. The compositions described herein may also be used in a method for the stimulation of collagen production, fibroblast production, fibrocyte production, or any combination thereof, comprising administration of a podiatric filler composition. The stimulation of collagen production, fibroblast production, fibrocyte production or any combination thereof, may be beneath the podiatric defect by administration of a podiatric filler composition.

The invention also provides for a method of restoring the functionality of the fat pads of the foot comprising administration of an effective amount of a podiatric filler composition. The method may include any further (including periodical or occasional) injections of said solution into such fat pads that may be necessary for long-term treatments.

The invention also provides for a method for restoring the thickness of atrophic damaged or degenerated fat-pads of the foot comprising administration of an effective amount of a podiatric filler composition. The method may comprise: (a) injecting a solution in the sub-calcaneal (heel), outside arch, or metatarsal (ball) fat pads of the foot; (b) restoring first the thickness of the natural pad; and/or (c) providing a durable thickness increase to a fat pad.

The compositions described herein may be administered to a fat pad. The fat pad may be located in the foot including one or any combination of the three fat pads in the human foot (e.g., under the heel, on the metatarsal head, and on the outer arch contact area.)

The methods described herein provides long-term reduction of a podiatric condition of a duration of at least about 1, 2, 3, 4, 5, 6, 7, 8, 9, or 10 years. The duration may be at least about 1-5, 3-5, or 1-10 years. The duration may also be at least about 10 years or longer.

The podiatric filler composition may be administered to the fat pads by injecting from a needle/syringe system. A 34-, 33-, 32-, 31-, 30-, 29-, 28-, 27-, 26-, 25-, 24-, 23-, 22-, 21-, 20-, 19-, 18-, 17-, 16-, 15-, 14-, 13-, 12-, 11-, 10-, 9-, 8-, or 7-gauge needle may be used. A standard length needle (2-3 cm) may be used. For example, a 30-, 27-, or 26-gauge needle of 0.5 inch length may be used. Any devices that enable one to percutaneously administer the solution to the fat pads may be used. For example, the podiatric filler composition may be injected using any devices designed for administering injectable fillers.

If required, a second injection of the podiatric filler composition may be administered after a period of at least about 1-4 weeks to 1-4 months. This additive effect may be used to achieve the desired result. More severely damaged foot fat pads may require a second, third and fourth injection of the podiatric filler composition.

Side-effects of superficial injection may be treated with corticosteroid cream or intrapodiatric corticosteroid injections. Dermabrasion will remove intrapodiatric granules of microbeads or microspheres. If microbeads or microspheres are used, the microbeads or microspheres can be spread around through massage within a day or two after the injection to even the distribution.

A colored dye may be included in the podiatric filler composition to allow the clinician to monitor the spread of the composition and its administration. Generally, the injection site may be evenly massaged and slight pressure may be applied to smooth out any detected lumps. Following massaging, this injection procedure consistently resulted in having the dye spreading in the entire pads, without dispersing into the other tissues. This confirms the interconnectivity of the fat pads' trabaecular structure, and demonstrates their confinement.

### Podiatric Conditions

Treatment of podiatric conditions must strike a balance between achieving long-term results, minimizing side effects or complications of the treatment procedure and the host tissue reaction thereto, and decreasing the recurrence of treatment to achieve the intended results.

The present invention provides compositions and methods for treating a variety of podiatric conditions including but not limited to arthritis (e.g., rheumatoid arthritis (RA)), bunions, fat pad atrophy, hammertoe (e.g., flexible hammertoes, rigid hammertoes), injury (e.g., trauma), metatarsalgia, neuroma, neuropathic pain, pain (e.g., heel pain), plantar faciitis, and podiatric defects (e.g., Haglund's deformity). The methods described herein also may provide long-term reduction of a podiatric defect, provide relief from pain, inflammation, and other podiatric conditions.

The present invention provides compositions and methods for treating a patient with neuropathic pain in the foot. The neuropathic pain in the foot may be due to a variety of etiologies include but are not limited to diabetic neuropathy, nerve injury, nerve tract injury, peripheral neuropathy, chemotherapy-induced neuropathy, chemotherapy-induced peripheral neuropathy, HIV-treatment induced neuropathy, HIV-treatment induced neuralgia, neuralgia, polyneuropathy, mononeuropathy, mononeuritis multiplex, autonomic neuropathy, symmetrical peripheral neuropathy, radiculopathy, large fiber peripheral neuropathy, small fiber peripheral neuropathy, and idiopathic neuropathic pain.

The present invention further provides compositions and methods for treating a patient with pain in the foot. The foot pain may be due to a variety of etiologies include but are not limited to chronic inflammatory pain, pain associated with arthritis, fibromyalgia, cancer-associated pain, chemotherapy-induced neuropathy, chemotherapy-induced peripheral neuropathy, pain associated with autoimmune disease, pain associated with hypertension, pain associated with cardiovascular disease, pain associated with rheumatic heart disease, pain associated with ulceration, pain associated with endocrine disease, pain associated with shingles or herpes zoster, spontaneous pain, chronic post-surgical pain, causalgia, postherpetic neuralgia, AIDS-related pain, complex regional pain syndromes type I and II, pain associated with spinal cord injury, and recurrent acute pain.

The present invention still further provides compositions and methods for treating a podiatric deformity that requires re-contouring, such as a small tissue defect (e.g., after animal bite(s)) or a deformity related to trauma where the deformity is cosmetically unappealing. In a further embodiment of the method of augmenting soft tissue, the augmentation may be after plastic surgery to achieve symmetry or a desired result.

Metatarsalgia refers to pain in the area between the arch and toes, or ball of the foot. The pain usually centers on one or more of the five bones (metatarsals) in this mid-portion of the foot. Also known as dropped metatarsal heads, metatarsalgia can cause abnormal weight distribution due to overpronation. Metatarsalgia occurs when one of the metatarsal joints becomes painful or inflamed. Metatarsalgia also can be caused by arthritis, foot injury (e.g., sports, a car accident, or repeated stress), hard surfaces (e.g., cement or tile floors), and specific footwear (e.g., rigid-soled work boots).

Plantar fasciitis—an inflammation on the bottom of the foot that leads to heel and/or arch pain. A variety of foot injuries or improper foot mechanics can lead to plantar fasciitis.

The methods described herein may also be used in treating pain or discomfort that occurs after treatment, such as surgery (e.g., removal of bunions) or physical therapy.

In accordance with further embodiments of the present invention, the present invention contemplates the use of any of the compositions described herein with other suitable compositions, including as mixtures or as separately administered substances, as would be recognized by persons skilled in the art based upon the guidance provided herein. According to further embodiments, the present invention contemplates the use of the compositions as medical devices or in conjunction with suitable medical devices, including implantable and non-implantable medical devices as would be understood by persons skilled in the art. The present invention aims at restoring fat pads of the foot by injecting an appropriate solution into such fat pads.

Further embodiments of the present invention will now be described with reference to the following examples. The examples contained herein are offered by way of illustration and not by any way of limitation.

### EXAMPLES

### Example 1

### Treatment of Metatarsalgia

A patient presenting with metatarsalgia is treated with injections of a podiatric filler composition comprising acrylate/methacrylate (A/M) copolymer powder (e.g., microspheres) and a cross-linked sodium hyaluronate.

The plantar surface of the patient's foot is washed with soap, rinsed with water, dried, and prepared with 70% isopropyl alcohol and a sterile gauze wipe. The site of injection may first be anaesthetized with an appropriate local anesthetic (e.g., Mepivacaine 3%).

A 27-gauge needle of 0.5 inch length syringe containing 0.5 cc (mL) of the podiatric filler composition may be used. Needle patency is verified by gently squeezing some of the podiatric filler composition out of the needle tip.

The foot fat pads can be reached by inserting a needle about 1 cm below the skin surface at the three main pressure points of the foot. The needle first goes through the thick plantar dermis before reaching the softer underlying fat pads. The fat pad under the heel is 1.6-2.0 cm thick. There is a small (about 0.5 mm thick) synovial burse between the pad and the calcaneum—which should be avoided. After the composition is administered, the patient may be evaluated to see if this compartment has been inadvertently punctured. There is no burse at the two other pad sites. For the heel site, this injection site is directly above the calcaneus, where heel spur normally develops. The clinician can feel the increased resistance in the syringe as the fat pad becomes refilled. The injected area may be evenly massaged and slight pressure may be applied to smooth out any detected lumps.

The patient is monitored for the development. If a pain continues, or reoccurs, appears during this time, the patient may be administered a subsequent effective amount of podiatric filler composition. The patient is monitored and treated according to the lessening of the symptoms and/or the development of side effects.

### Example 2

### Treatment of Fat Pad Atrophy

A patient presenting with fat pad atrophy is treated with injections of a podiatric filler composition comprising acrylate/methacrylate (A/M) copolymer powder (e.g., microspheres) and collagen.

The plantar surface of the patient's foot is washed with soap, rinsed with water, dried, and prepared with 70% isopropyl alcohol and a sterile gauze wipe. The site of injection may first be anaesthetized with an appropriate local anesthetic (e.g., Mepivacaine 3%).

A 27-gauge needle of 0.5 inch length syringe containing 0.5 cc (mL) of the podiatric filler composition may be used. Needle patency is verified by gently squeezing some of the podiatric filler composition out of the needle tip.

The foot fat pads can be reached by inserting a needle about 1 cm below the skin surface at the three main pressure points of the foot. The needle first goes through the thick plantar dermis before reaching the softer underlying fat pads. The fat pad under the heel is 1.6-2.0 cm thick. There is a small (about 0.5 mm thick) synovial burse between the pad and the calcaneum—which should be avoided. After the composition is administered, the patient may be evaluated to see if this compartment has been inadvertently punctured. There is no burse at the two other pad sites. For the heel site, this injection site is directly above the calcaneus, where heel spur normally develops. The clinician can feel the increased resistance in the syringe as the fat pad becomes refilled. The injected area may be evenly massaged and slight pressure may be applied to smooth out any detected lumps.

The patient is monitored for the development. If a pain continues, or reoccurs, appears during this time, the patient may be administered a subsequent effective amount of podiatric filler composition. The patient is monitored and treated according to the lessening of the symptoms and/or the development of side effects.

### Example 3

### Treatment of Plantar Fasciitis

A patient presenting with plantar fasciitis is treated with injections of a podiatric filler composition comprising acrylate/methacrylate (A/M) copolymer powder (e.g., microspheres) and collagen.

The plantar surface of the patient's foot is washed with soap, rinsed with water, dried, and prepared with 70% isopropyl alcohol and a sterile gauze wipe. The site of injection may first be anaesthetized with an appropriate local anesthetic (e.g., Mepivacaine 3%).

A 27-gauge needle of 0.5 inch length syringe containing 0.5 cc (mL) of the podiatric filler composition may be used. Needle patency is verified by gently squeezing some of the podiatric filler composition out of the needle tip.

The foot fat pads can be reached by inserting a needle about 1 cm below the skin surface at the three main pressure points of the foot. The needle first goes through the thick plantar dermis before reaching the softer underlying fat pads. The fat pad under the heel is 1.6-2.0 cm thick. There is a small (about 0.5 mm thick) synovial burse between the pad and the calcaneum—which should be avoided. After the composition is administered, the patient may be evaluated to see if this compartment has been inadvertently punctured. There is no burse at the two other pad sites. For the heel site, this injection site is directly above the calcaneus, where heel spur normally develops. The clinician can feel the increased resistance in the syringe as the fat pad becomes refilled. The injected area may be evenly massaged and slight pressure may be applied to smooth out any detected lumps.

The patient is monitored for the development. If a pain or inflammation continues, or reoccurs, appears during this time, the patient may be administered a subsequent effective amount of podiatric filler composition. The patient is monitored and treated according to the lessening of the symptoms and/or the development of side effects.

### Example 4

### Treatment of Foot Pain

A patient presenting with foot pad pain which has been resistant to prior treatment. The patient is treated with injections of a podiatric filler composition comprising acrylate/methacrylate (A/M) copolymer powder (e.g., microspheres) and collagen.

The plantar surface of the patient's foot is washed with soap, rinsed with water, dried, and prepared with 70% isopropyl alcohol and a sterile gauze wipe. The site of injection may first be anaesthetized with an appropriate local anesthetic (e.g., Mepivacaine 3%).

A 27-gauge needle of 0.5 inch length syringe containing 0.5 cc (mL) of the podiatric filler composition may be used. Needle patency is verified by gently squeezing some of the podiatric filler composition out of the needle tip.

The foot fat pads can be reached by inserting a needle about 1 cm below the skin surface at the three main pressure points of the foot. The needle first goes through the thick plantar dermis before reaching the softer underlying fat pads. The fat pad under the heel is 1.6-2.0 cm thick. There is a small (about 0.5 mm thick) synovial burse between the pad and the calcaneum—which should be avoided. After the composition is administered, the patient may be evaluated to see if this compartment has been inadvertently punctured. There is no burse at the two other pad sites. For the heel site, this injection site is directly above the calcaneus, where heel spur normally develops. The clinician can feel the increased resistance in the syringe as the fat pad becomes refilled. The injected area may be evenly massaged and slight pressure may be applied to smooth out any detected lumps.

The patient is monitored for the development. If a pain continues, or reoccurs, appears during this time, the patient may be administered a subsequent effective amount of podiatric filler composition. The patient is monitored and treated according to the lessening of the symptoms and/or the development of side effects.

### Example 5

### Treatment of Diabetic Patient with Foot Pain

A diabetic patient complaining of chronic foot pad is examined and then treated with injections of a podiatric filler composition comprising acrylate/methacrylate (A/M) copolymer powder (e.g., microspheres) and collagen.

The plantar surface of the patient's foot is washed with soap, rinsed with water, dried, and prepared with 70% isopropyl alcohol and a sterile gauze wipe. The site of injection may first be anaesthetized with an appropriate local anesthetic (e.g., Mepivacaine 3%).

A 27-gauge needle of 0.5 inch length syringe containing 0.5 cc (mL) of the podiatric filler composition may be used. Needle patency is verified by gently squeezing some of the podiatric filler composition out of the needle tip.

The foot fat pads can be reached by inserting a needle about 1 cm below the skin surface at the three main pressure points of the foot. The needle first goes through the thick plantar dermis before reaching the softer underlying fat pads. The fat pad under the heel is 1.6-2.0 cm thick. There is a small (about 0.5 mm thick) synovial burse between the pad and the calcaneum—which should be avoided. After the composition is administered, the patient may be evaluated to see if this compartment has been inadvertently punctured. There is no burse at the two other pad sites. For the heel site, this injection site is directly above the calcaneus, where heel spur normally develops. The clinician can feel the increased resistance in the syringe as the fat pad becomes refilled. The injected area may be evenly massaged and slight pressure may be applied to smooth out any detected lumps.

The patient is monitored for the development. If a pain continues, or reoccurs, appears during this time, the patient may be administered a subsequent effective amount of podiatric filler composition. The patient is monitored and treated according to the lessening of the symptoms and/or the development of side effects.

Although the invention has been described in some detail by way of illustration and example for purposes of clarity of understanding, it will be obvious that certain changes and modifications may be practiced within the scope of the appended claims. Modifications of the above-described modes for carrying out the invention that are obvious to persons of skill in medicine, pharmacology, and/or related fields are intended to be within the scope of the following claims.

All publications (e.g., Non-Patent Literature), patent application publications, and patent applications mentioned in this specification are indicative of the level of skill of those skilled in the art to which this invention pertains. All such publications (e.g., Non-Patent Literature), patent application publications, and patent applications are herein incorporated by reference to the same extent as if each individual publication, patent, patent application publication, or patent application was specifically and individually indicated to be incorporated by reference.

What is claimed is:

1. A method for treating or preventing a condition in a subject's foot comprising injecting into a fat pad of the subject's foot an effective amount of a composition comprising an unsubstituted acrylate/substituted acrylate copolymer.

2. The method of claim 1, wherein the condition is fat pad atrophy, foot pain, plantar faciitis, metatarsalgia, or foot injury.

3. The method of claim 1, wherein the condition is a progressive orthopedic foot deformity.

4. The method of claim 3, wherein the progressive orthopedic foot deformity is pes cavus, digitii flexus, equinous deformity or any combination thereof.

5. The method of claim 1, wherein the subject has a systemic disease.

6. The method of claim 5, wherein the systemic disease is rheumatoid arthritis.

7. The method of claim 1, wherein the subject has diabetes.

8. The method of claim 1, wherein the condition is a shift of the fat pad under the metatarsal heads.

9. The method of claim 8, wherein the shift of the fat pad under the metatarsal heads is caused by the subject wearing high heeled shoes.

10. The method of claim 1, wherein the suspension has a viscosity of about 100 to about 1,000 mPa·S.

11. The method of claim 1, wherein the unsubstituted acrylate/substituted acrylate copolymer comprises particles in a suspension.

12. The method of claim 11, wherein the particles have a diameter of less than about 100 μm.

13. The method of claim 11, wherein the particles have a diameter of less than about 10 μm

14. The method of claim 11, wherein the particles have a diameter of about 0.01 μm to about 10 μm.

15. The method of claim 11, wherein the particles have a diameter of about 0.1 μm to about 5 μm.

16. The method of claim 1, wherein the substituted acrylate monomer of the unsubstituted acrylate/substituted acrylate copolymer is substituted with a methyl group.

17. The method of claim 1, wherein the substituted acrylate monomer is substituted with a hydrocarbon chain of two, three, four or five carbons.

18. The method of claim 1, wherein the substituted acrylate monomer is substituted with a halogen group.

19. The method of claim 1, wherein the substituted acrylate monomer is substituted with a nitrile group.

20. The method of claim 1, wherein the acrylate/methacrylate copolymer is a finely ground solid.

21. The method of claim 20, wherein the finely ground solid has a diameter of from about less than 0.1 μm to about 10 μm.

22. The method of claim 1, wherein the composition further comprises a physiologically acceptable suspending agent.

23. The method of claim 22, wherein the physiologically acceptable suspending agent is resorbable.

24. The method of claim 22, wherein the physiologically acceptable suspending agent is a buffered physiological solution.

25. The method of claim 22, wherein the physiologically acceptable suspending agent comprises cross-linked sodium hyaluronate.

26. The method of claim 22, wherein the physiologically acceptable suspending agent comprises a non cross-linked sodium hyaluronate.

27. The method of claim 22, wherein the physiologically acceptable suspending agent comprises collagen.

28. The method of claim 1, further comprising administering an anti-inflammatory agent, an analgesic, a local anesthetic or a combination thereof.

29. A method for treating or preventing a condition in a subject's foot comprising injecting into a fat pad of the subject's foot an effective amount of a composition comprising a copolymer of phenylethyl acrylate and phenylethyl methacrylate.

30. The method of claim 29, wherein the composition further comprises a physiologically acceptable suspending agent.

31. The method of claim 29, further comprising administering an anti-inflammatory agent, an analgesic, a local anesthetic or a combination thereof.

32. A method for supplementing a fat pad of a subject comprising injecting into a fat pad of the subject an effective amount of a composition comprising an unsubstituted acrylate/substituted acrylate copolymer.

33. The method of claim 32, wherein the substituted acrylate monomer of the unsubstituted acrylate/substituted acrylate copolymer is substituted with a methyl group.

34. The method of claim 32, wherein the substituted acrylate monomer is substituted with a hydrocarbon chain of two, three, four or five carbons.

35. The method of claim 32, wherein the substituted acrylate monomer is substituted with a halogen group.

36. The method of claim 32, wherein the substituted acrylate monomer is substituted with a nitrile group.

37. The method of claim 32, wherein the unsubstituted acrylate/substituted acrylate copolymer is a copolymer of phenylethyl acrylate and phenylethyl methacrylate.

38. A method of preparing a composition for treating or preventing a condition in a subject's foot comprising admixing a finely ground copolymer of phenylethyl acrylate and phenylethyl methacrylate with a physiologically acceptable suspending agent to form an injectable, biocompatible material.

39. A composition for treating fat pad atrophy comprising an unsubstituted acrylate/substituted acrylate copolymer in an effective amount for treating fat pad atrophy and a physiologically acceptable suspending agent.

40. The composition of claim 39, further comprising an anti-inflammatory agent, an analgesic, a local anesthetic or a combination thereof.

41. A composition for treating fat pad atrophy comprising a proteoglycan and a physiologically acceptable suspending agent in an effective amount for treating fat pad atrophy.

42. The composition of claim 41, wherein said proteoglycan is aggrecan, decorin, biglycan, fibromodulin, lumican, keratocan, epiphycan, or osteoglycin.

43. The composition of claim 41, further comprising an anti-inflammatory agent.

44. The composition of claim 43, wherein said anti-inflammatory agent is a non-steroidal anti-inflammatory drug (NSAID).

45. The composition of claim 43, wherein said anti-inflammatory agent is acetaminophen, azapropazone, diclofenac, diflunisal, etodolac, fenbufen, fenoprofen, flurbiprofen, ibuprofen, indomethacin, ketoprofen, ketorolac, mefenamic, meloxicam, nabumetone, naproxen, phenylbutazone, piroxicam, a salicylate, sulindac, tenoxicam, tiaprofenic acid, or tolfenamic acid.

46. The composition of claim 45, wherein said salicylate is acetylsalicylic acid, amoxiprin, benorylate, choline magnesium salicylate, ethenzamide, faislamine, methyl salicylate, magnesium salicylate, salicyl salicylate, or salicylamide.

47. The composition of claim 41, further comprising administering an analgesic.

48. The composition of claim 47, wherein said analgesic is acetaminophen, amitriptyline, carbamazepine, codeine, dihydromorphine, fentanyl patch, Flupirtine, fluriprofen, gabapentin, hydrocodone APAP, hydromorphone, ibuprofen, ketoprofen, morphine, oxycodone, pentazocine, pethidine, phenacetin, pregabalin, propoeylphene, propoyl APA, salicylamide, tramadol, tramadol APAP, or voltaren.

49. The composition of claim 41, further comprising a local anesthetic.

50. The composition of claim 49, wherein said local anesthetic is amethocaine, articaine, benzocaine, bupivacaine, mepivacaine, cocaine, cinchocaine, chloroprocaine, cyclomethycaine, dibucaine, dimethocaine, EMLA® (eutectic mixture of lidocaine and prilocalne), etidocaine, larocaine, levobupivacaine, lidocaine, lignocaine, procaine, piperocaine, prilocalne, proparacaine, propoxycaine, ropivacaine, saxitoxin, tetracaine, tetrodotoxin, or trimecaine.
